(19) United States
(12) Patent Application Publication
(10) Pub. No.: US 2022/0110589 A1
(43) Pub. Date: Apr. 14, 2022

(54) Disposable device for remote biomedical monitoring

(71) Applicants: Adrian Rica Vicente, Madrid (ES); Francisco Paul Mikuski Silva, Madrid (ES)

(72) Inventors: Adrian Rica Vicente, Madrid (ES); Francisco Paul Mikuski Silva, Madrid (ES)

(21) Appl. No.: 17/065,545

(22) Filed: Oct. 8, 2020

(51) Int. Cl. A61B 5/00; A61B 5/01; A61B 5/1455; A61B 7/04; A61B 5/11

(52) U.S. Cl. CPC A61B 5/6833; A61B 5/01; A61B 5/14552; A61B 7/04; A61B 2560/0214; A61B 5/746; A61B 5/11; A61B 2562/0271; A61B 5/002

(57) ABSTRACT

A disposable remote biomedical monitoring device made up of a body in the form of a patch, which adheres directly to the skin of people by means of a Biocompatible glue said patch comprises, housed in a body of plastic material a biocompatible and disposable plastic consisting of a base and a closing cap and containing a temperature sensor, a reflex pulse oximetry sensor and a digital microphone linked to a printed circuit with non-volatile memory connected, in turn, to an electrical power supply battery and a Bluetooth® communication module that connects, wirelessly, with a specific App implemented on a smartphone, PC or similar device of the medical health personnel or the user himself to view and manage the registered parameters.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### STATEMENT REGARDING PRIOR DISCLOSURES BY THE INVENTOR OR A JOINT INVENTOR

Not Applicable

### BACKGROUND OF THE INVENTION

### Field of the Invention

The field of application of the present invention is framed within the sector of the industry dedicated to the manufacture of medical devices for body monitoring.

### Description of Related Art

As is known, some diseases and, especially, the COVID-19 coronavirus are highly contagious even in asymptomatic cases, which is why it is necessary to apply strict isolation measures, both for hospitalized patients, whether in the plant or in the ICU, as for those who do not present symptoms but who, likewise, must be kept under certain medical surveillance to detect any worsening of their condition. This means that, in one case as in another, the medical and health professionals who take care of these patients must protect themselves with full EPIS every time they have to attend to them and, even so, are continuously at risk due to the need to come into contact with them to take the temperature, auscultate them and perform other essential control operations to maintain a control of the condition of these patients.

The objective of the present invention is, therefore, to provide a means to avoid, at least in certain routine actions such as those mentioned above taking the temperature, heart pulse and blood oxygen level, having to carry them out directly in contact with the patient and being able to carry them out remotely through the use of wireless communication technology and computer science, for example from an adjacent room or room, thus minimizing the possibility of transmission and saving time and protective equipment.

On the other hand, and as a reference to the current state of the art, it should be noted that, at least by the applicant, the existence of any other disposable remote biomedical monitoring device, or any other invention with a similar application, technical and structural characteristics equal or similar to those presented by the one claimed herein, is unknown.

### BRIEF SUMMARY OF THE INVENTION

The invention, as expressed in the wording of the present specification, refers to a disposable remote biomedical monitoring device that provides, for its intended function, advantages and characteristics, which are described in detail below.

More specifically, the object of the invention focuses on an electronic device that, consisting of a small patch, about 30 mm in diameter, that adheres to the skin of people by means of a biocompatible glue, mainly intended for use in people sick with COVID-19 or other asymptomatic highly infectious diseases and sick people hospitalized or in intensive care, allows monitoring the evolution of body temperature and other basic biomedical parameters such as the amount of oxygen in the blood and the heart rate of said people by part of the medical or health personnel, from the moment the device is activated, remotely, that is, without the need to come into physical contact with them, since it includes, at least, a temperature sensor, a reflex pulse oximetry sensor and a digital microphone linked to a printed circuit connected to a power battery and a Bluetooth® communication module that c connect with an App implemented on a smartphone, pc or similar device.

### BRIEF DESCRIPTION OF THE DRAWINGS

To complement the description that is being made and in order to help a better understanding of the characteristics of the invention, the present specification is accompanied, as an integral part thereof, by a set of drawings in which for illustrative purposes and non-limiting, the following has been represented:

FIG. 1.—Shows a schematic perspective view of an embodiment of the patch that constitutes the disposable remote biomedical monitoring device object of the invention, showing:
1: disposable patch
2: base
2a: cover
3: extension wing

FIG. 2.—is a front view of the device showing:
1: disposable patch
2: base
2a: cover
3: extension wing
4: one body temperature sensor
5: reflex pulse oximetry sensor
6: digital microphone
7: printed circuit with non-volatile memory
8: battery
9: Bluetooth® communication module
10: movement and verticality sensor
11: presence detector

FIG. 3.—Shows a representation of the placement of the patch on the user's body, directly on the skin in the heart area.

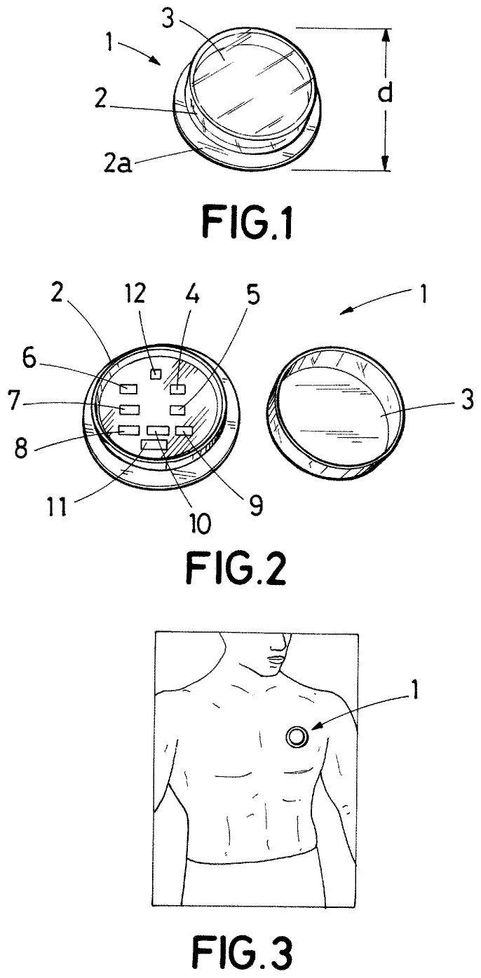

### DETAILED DESCRIPTION OF THE INVENTION

The disposable remote biomedical monitoring device that the invention proposes enables the aforementioned objectives to be satisfactorily achieved, the characterizing details that make it possible and that distinguish it are conveniently set out in the final claims that accompany the present description.

What the invention proposes, as noted above, is a small electronic device that, made up of a body in the form of a patch, about 30 mm in diameter, which adheres directly to the skin of people by means of a Biocompatible glue, and that can be detached by means of a biocompatible liquid, being mainly intended for use in people sick with COVID-19 or other highly infectious diseases, whether they are asymptomatic or are sick people hospitalized or in intensive care, has as The purpose is to be able to monitor remotely, that is to say at a distance and without the need to come into physical contact with them, the evolution of body temperature and other basic biomedical parameters, such as the amount of oxygen in the blood and the heart pulse, of these people by medical or health personnel, from the moment the device is activated, minimizing the risk of contagion and saving time and protection material.

For this, said patch comprises, housed in a body of plastic material A biocompatible and disposable plastic consisting of a base and a closing cap being at least one body temperature sensor, a reflex pulse oximetry sensor and a digital microphone linked to a printed circuit with non-volatile memory connected, in turn, to an electrical power supply battery and a Bluetooth® communication module that connects, wirelessly, with a specific App implemented on a smartphone, PC or similar device of the medical health personnel or the user himself to view and manage the registered parameters.

The pulse oximetry sensor by reflection allows the detection of the amount of oxygen in the blood together with the detection of the heart pulse. And, for its part, the digital microphone allows remote auscultation of the heart and lungs through the Bluetooth® communication channel.

In addition, the device allows, eventually, the remote auscultation of the heart and lungs in an intelligent way, using artificial intelligence, that is, by linking to specific software the system may be able to detect pathologies of the heart muscle, pulmonary pulse, and other conditions such as and as is the case of detection in its early stages of pathologies related to lung function, as is the case of processes related to pneumonia.

Thus, the device is ideal for treating two specific different types of patients:
Asymptomatic people, who are infected, testing positive for COVID-19 or another type of virus or contagious bacteria, who must remain in confinement because they do not present symptoms that warrant their confinement in the clinic,
and infected people who present mild or severe symptoms, who require specialized care such as in a hospital confinement, or in places of treatment such as medicalized hotels or similar institutions.

Optionally, the patch can also comprise a motion sensor and/or a verticality sensor, to monitor the time that the patient remains at rest or lying down, making it possible to assume that they have symptoms of disease.

Likewise, it is also contemplated that, optionally, the device comprises a sensor or detector of other similar devices from a certain distance, to alert of the proximity between users.

On the other hand, preferably the battery that the device has is a small button cell with an autonomy of at least one month operating continuously, which can optionally be replaced once it is exhausted.

Preferably, the device has electronics that are only activated by holding it in the hand and hitting it against a surface and then by detecting the user's body temperature.

Optionally, the device also includes an operation indicator LED, which lights up when the device is activated.

With all this, the device of the invention allows continuous monitoring functions of important biomedical parameters such as:
Non-contact body temperature. Accurate monitoring of body temperature with its record in non-volatile memory. This, in a way and form that health personnel can study its evolution remotely.
Amount of oxygen in the blood. Measurement of oxygen in blood with the emission of alarms in real time to the GPs as they are established by their own criteria remotely.
Heartbeat. Measurement of the heart pulse with the emission of alarms in real time to the GPs according to their remotely established criteria.
Verticality or motility. Measurement of the horizontality parameter that determines how long the person uses the bed. This also includes the mobility index of people during their confinement.
Intelligent remote auscultation. Remote auscultation of the functioning of the heart and lungs in a way similar to the use of a stethoscope but audible from the Smartphone of the doctors themselves.
Location control. Optionally, it allows tracking by geo-positioning, if people allow it, in this case using the GPS locator of the user's Smartphone.

The device also allows that, using the user's own telephone, the alarms generated are transferred to their GPs so that they can make the decision to keep the infected person at home or take them to hospitalization.

For its part, the advantages it provides are numerous, highlighting the following:
Low cost system to monitor people remotely, without the need for physical contact or approach.
It is disposable totally watertight and made of biocompatible material and includes an internal battery with a duration of at least one month
It can warn when the user has a fever and monitors vital parameters remotely for use by medical health personnel, who can set the alarm levels on these parameters using an App installed on their mobile phones or Smartphone.
Works in real time so that alarms can be viewed as they occur.
Use Bluetooth® 5.0 type wireless communication with a minimum range of 20 meters with the phone that has the APP.
It does not require post-processing platforms of the information to function, although this possibility is available and is not necessary since the alarm information is directed directly to the treating doctors.
It greatly reduces the possibility of infection of medical health personnel due to the fact that close contact with infected people is not necessary to measure their vital signs.
It makes it possible to free health personnel from work in hospitals and seclusion or isolation centers for quarantine, since a single person of health personnel can attend to many hospitalized people with less effort in real time, which does not currently occur with the available care protocols.
Allows real-time monitoring of large numbers of people automatically, especially in the case of nursing homes for the elderly, medical health personnel in clinics, hospitals, institutions. With this type of monitoring, infections between asymptomatic people are reduced to a minimum.
Stores in non-volatile memory all the biomedical parameters of the people with respect to the moment in which it was activated. This makes it possible to carry out a posteriori Big data type studies that would help in the analysis of diseases and pandemics.
It allows the geolocation of asymptomatic people, which allows the health monitoring of infected people in order to obtain information on possible infections according to geo-positioning.
It allows the detection of proximity with other patches to know if the safety distance between the people who use the patches is maintained to detect the proximity of the medical health personnel in order to detect their attention to the patient or also detect the exposure of the same to a possible infected.
It allows connecting via Bluetooth® with artificial intelligence processors to be able to carry out diagnoses on heart and pulmonary pathologies such as the case of detection of pneumonia processes in its early stages, heart arrhythmias, and remote auscultation by means of audio using any Smartphone from the market.

### Description of the Preferred Embodiment

In view of the aforementioned figures, and in accordance with the adopted numbering, an example of a non-limiting embodiment of the disposable remote biomedical monitoring device of the invention can be observed, which comprises what is indicated and described in detail below.

Thus, as seen in said figures, the device in question is an electronic device that comprises a hollow body in the form of a disposable patch (1), about 30 mm in diameter (d), preferably formed by a base (2) and a cover (3), and which has, on the external part of said base (2), where it has a perimeter wing (2a) to enlarge its surface, a biocompatible glue to adhere it to the skin of a patient/user, defining a watertight housing that, in turn, comprises at least one body temperature sensor (4), a reflex pulse oximetry sensor (5) and a digital microphone (6) linked to a printed circuit (7) with memory non-volatile connected, in turn, to a battery (8) and a Bluetooth® communication module (9), also housed in said patch (1), which connects, wirelessly, with a specific App implemented on a smartphone, pc or device

Analogue device (not shown) capable of supporting it and managed by medical healthcare personnel and/or the patient/user himself, where the data recorded by said sensors can be viewed, monitored and managed, as well as set alert parameters on them, as appropriate.

Optionally, the patch (1) further comprises a movement sensor and/or a verticality sensor (10), to monitor the time that the patient remains at rest or lying down, making it possible to assume that they have symptoms of disease.

Optionally, the patch (1) also comprises a presence detector (11) of other similar devices from a certain distance, to alert of the proximity between users.

In any case, preferably, the battery (8) is a button cell with autonomy of at least one month operating continuously which, optionally, can be replaceable once exhausted.

Preferably, the body of the patch (1) is made of biocompatible plastic material.

Preferably, the electronics of the patch (1) comprise means of activation by shaking linked to a minimum of temperature detected by the temperature sensor (4), once it has been adhered to the skin thereof.

Optionally, the patch (1) also comprises an operation indicator led (12), which turns on when the device is activated.

Having sufficiently described the nature of the present invention, as well as the way of putting it into practice, it is not considered necessary to make its explanation more extensive so that any expert in the field understands its scope and the advantages derived from it, stating that, Within its essentiality, it may be put into practice in other forms of implementation that differ in detail from the one indicated by way of example, and to which it will also achieve the protection that is sought provided that its fundamental principle is not altered, changed or modified.

1. A disposable electronic device for remote biomedical monitoring comprising
a base with a biocompatible glue on one of its sides to adhere it to the skin of a patient/user,
a watertight housing that comprises
a body temperature sensor
a reflex pulse oximetry sensor
a digital microphone
a printed circuit with a non-volatile memory connected to
a battery
a Bluetooth® communication module connected with a specific application implemented on a smartphone, pc or similar device capable of supporting it where data can be viewed, monitored and managed as registered by the sensors to establish alert parameters.
a cover.

2. The device according to claim 1, wherein the device is of round shape and 30 mm in diameter.

3. The device according to claim 1, wherein the base has an extension wing to expand its surface.

5. The device according to claim 1, wherein the device also comprises a movement sensor and a verticality sensor.

6. The device according to claim 1, wherein the device comprises a presence detector to detect other similar devices from a certain distance.

7. The device according to claim 1, wherein the battery is a button cell with autonomy of at least one month operating continuously.

8. The device according to claim 1, wherein the battery is replaceable once exhausted.

9. The device according to claim 1, wherein the body of the device is made of biocompatible plastic material.

10. The device according to claim 1, wherein the electronics of the device comprise vibratory means of activation linked to a minimum temperature setting detected by the temperature sensor.
